(19) United States
(12) Patent Application Publication
(10) Pub. No.: US 2012/0121717 A1
(43) Pub. Date: May 17, 2012

(54) DRUG-LOADED POLYSACCHARIDE-COATED GOLDMAG PARTICLES (DPGPs) AND ITS SYNTHESIS METHOD

(75) Inventors: Xu Chao, Shaanxi (CN); Yali Cui, Shaanxi (CN); Mingli Peng, Shaanxi (CN); Chao Chen, Shaanxi (CN); Xiaofang Xin, Shaanxi (CN); Ke Li, Shaanxi (CN); Wenli Hui, Shaanxi (CN)

(73) Assignee: XI'AN GOLDMAG NANOBIOTECH CO. LTD

(21) Appl. No.: 13/142,820

(22) PCT Filed: Dec. 30, 2008
(86) PCT No.: PCT/CN2008/002130

§ 371 Date: Sep. 21, 2011

(30) Foreign Application Priority Data
Dec. 30, 2008 (CN) 200810236540.4

(51) Int. Cl. A61K 9/14; B05D 3/00; A61P 31/00; A61K 31/704; A61P 35/00

(52) U.S. Cl. 424/493; 514/34; 427/2.14

(57) ABSTRACT

The invention relates to Polysaccharide-coated GoldMag particles (DPGPs) and the method of its synthesis, which characterized GoldMag particles as a core and natural or synthetic biodegradable polysaccharide such as dextran, cyclodextrin and derivatives as shell. DPGPs are synthesized by mixing Polysaccharide-coated GoldMag particles (DPGPs) with drug through physical bond. The preparation of the drug-loaded composite particles include: preparing the polysaccharide-coated GoldMag particles and then loading the drug on the polysaccharide-coated GoldMag particles. The drug-loading process is carried out through directly mixing the polysaccharide-coated GoldMag particles with the drug solution by the shaker. That means the polysaccharide-coated GoldMag particles load the drug through affinity adsorption.

### FIELD OF INVENTION

The invention relates to DPGPs and its synthesis method ,especially to drug-loaded composite particles using polysaccharide-modified GoldMag particles(PGPs), which has better biocompatibility and higher drug-loading rate. It characterized GoldMag particles as a core and natural or synthetic biodegradable polysaccharide such as dextran, cyclodextrin and derivatives as shell.

### BACKGROUND OF THE INVENTION

Drug-loaded magnetic microspheres, as the fourth generation targeting preparation, can increase drug efficacy meanwhile decrease side effects, and thus provide a new approach for the chemotherapy in the clinical treatment. They will be stable preparations produced by both anti-tumor drug and magnetic material, especially biocompatible biomacromolecules, embedded or adsorbed in polymeric material. When the preparation injected into the body, it gradually concentrates at the site of the tumor under an sufficiently strong external magnetic field. The drug carrier degrades through the catalysis of enzyme or varying physical-chemical conditions such as Ph value, osmotic pressure or temperature. So the drug can be released slowly, resulting in a high blood drug level at the site of the tumor and a low blood drug level in other sites of the body. This therapy could improve the efficiency of the treatment and reduce releasing rate and systemic toxicity.

Magnetic microspheres consist of magnetic material and carrier material. The magnetic material generally include pure iron powder, carbonyl iron, magnetite, Fe—Co alloy and so on, especially Fe₃O₄ magnetic fluid. The magnetic material should have a particle size as small as possible, typically 10-30 nm, and excellent magnetic property. Conventional modification material include natural polymer, such as albumin, gelatine, chitosan, dextran and starch; and synthetic polymer, such as polycarbonate, polyalkylcyanoacrylate, polyvinylpyrrolidone, polylactic acid and copolymers. They can reduce systemic toxicity, increase biocompatibility, and reduce clearance of endothelial cells. In addition, these difunctional materials can be absorbed to magnetic particles through chemical bonding or physical adsorption, and on the other hand they can absorb drug to delay and control the release of drug.

In 1996, Lübbe et al., carried out the first clinical treatment of targeted therapy using drug-loaded magnetic nanoparticles. In the magnetically targeted therapy, 14 patients with terminal solid tumor were treated, the result indicated the patients have good tolerance to magnetically drug-loaded magnetic nanoparticles. In 2002, Magnetically Targeted Carrier-Doxorubicin (MTC-DOX) technique developed by FeRx Inc., U.S. was approved by U.S. FDA, and mainly used for the treatment of liver cancer(hepatic cell carcinoma-HCC). In the annual meeting named “Molecular targeting and cancer therapy” in November, 2002, FeRx Inc. reported phase I/II clinical test results of targeted therapy of hepatic cell carcinoma, and predicted that this new technology had great potential in treating live cancer. Liang et al. studied the application of superparamagnetic iron oxide nanoparticles modified by amino group as a new biomagnetic targeted vehicle in magnetically targeted therapy for live cancer. Professor Zhang Yangde et al., a professor of Central South University, studied the therapy of transplanted liver cancer by magnetic albumin particles containing doxorubicin, and found that the magnetic albumin particles loaded doxorubicin have good therapeutic efficacy under an external magnetic field. Mu Rong studied the magnetically targeted therapy to the rat transplanted liver cancer using magnetic chitosan microspheres loaded with doxorubicin, and the result suggested that the magnetic chitosan microspheres loaded with drug have good targeted therapeutic efficacy. There are many reports concerning the application of gold nanoparticles in biomedical field. Priyabrata Mukherjee et al. coupled gold nanoparticles with vascular endothelial cell growth factors to treat chronic lymphocytic leukaemia. The results indicated that a certain dose of gold nanoparticles used alone did not lead to considerable cell apoptosis; however, gold nanoparticles coupled with vascular endothelial cells can lead to considerable apoptosis of cell. These findings confirmed the advantages of drug delivery system using gold nanoparticles in the treatment of human malignant disease. Giulio et al. studied the efficacy and toxicity of colloidal gold as vehicle carrying tumor-inhibiting factor in targeted drug delivery. The results indicated that colloidal gold has no significant harmful effect on cells, and the colloidal gold loaded with a tumor-inhibiting factor has significant lethal effect on tumor cells. Yao Cuiping et al. combined immunocolloidal gold with alkaline phosphatase specific antibody in bovine intestines and after irradiation with laser, to treat human malignant lymphadenoma cell Karpas 299. The results indicated that, after laser irradiation, Karpas 299 cells combined with gold particles have a mortality of more than 95%; however, KG₁ cells without gold particles hardly changed. This indicated that gold particles have no significant side effect on human cell line. Zharov et al. designed a system in which gold particles whose size was 40 nm were combined with antigens in MDA-MB-231 breast carcinoma cells via anti-bodies, and the combination of cells and particles were irradiated with laser. Then it was observed that a lot of gas bubbles formed around the nanoclusters and tumor cells died. El-Sayed et al. conducted primary studies of cancer cell diagnosis using immunocolloidal gold and obtained some achievements. The above studies indicate that, liver cancer targeted therapy using the combination of magnetic particles with gold nanoparticles can take advantage of both the magnetically targeted property of magnetic particles and the ability of gold element of enhancing non-specific immune reaction. The composite particles can be used for targeted drug delivery and at the same time enhance immunity. It has potent application prospect.

U.S. Pat. No. 7,226,636B2 reported a process of preparing gold-coated magnetic nanoparticles. The process includes the steps of synthesis comprising: in a ferrofluid suspension in a suitable liquid, adding an amount of reducible gold compound and reducing agent to the suspension, and finally maintaining the suspension for sufficient time to form gold-coated magnetic nanoparticles. UK patent GB 2415374A reported a gram-scale synthesis process of core-shell structure magnetic nanoparticles, in which core is γ-Fe₂O₃ and shell is gold. U.S. Pat. No. 7,232,471B2 reported the synthesis method of gold nanoparticles modified by cyclodextrin. Chinese Patent ZL 03124061.5 and ZL 03153486.4 disclosed the synthesis of core-shell structure and assembling type of gold-magnetic particles; however, further modification and their application in targeted drug delivery have not been reported.

The patent entitled “superparamagnetic drug-loaded body and its preparation” (application number :200610104757.0), which was filed by the present applicants in 2006, relates to the preparation of drug-loaded body using GoldMag particles and its use in targeted therapy. However, the claims of this patent are not precise and fail to define the parameters specifically. For example, it does not provide specific particle size, saturation magnetization, magnetic property, drug-loading rate and encapsulation rate of the superparamagnetic composite particles, and does not provide specific preparation process and physical-chemical parameters of the drug-loaded carrier using polysaccharide-coated GoldMag particles.

### SUMMARY OF THE INVENTION

### Aim of the Invention

To overcome the above-mentioned technical problems, the invention provides a superparamagnetic drug-loaded composite using polysaccharide-coated GoldMag particles, which exhibits good biocompatibility without side effects, and has certain particle size and good effects of delaying and controlling drug release. Its encapsulation rate fulfils the requirement of Chinese Pharmacopoeia. The invention also provides a process of preparation.

### Technical Solution of the Invention

The drug-loaded body using polysaccharide-coated GoldMag particles is characterized in that the synthesis of the drug composite is carried out by directly mixing polysaccharide-coated GoldMag particles with drug solution to load the drug on the GoldMag particles through physical adsorption; wherein said polysaccharide-coated GoldMag particles are synthesized by mixing assemble or core-shell structure GoldMag particles with natural or synthetic polysaccharide polymer to coat the GoldMag particles with the polymer through chemical bonding or physical adsorption; or said polysaccharide-coated GoldMag particles are formed by using GoldMag particles as the core and make polysaccharide molecules form reticular structures through cross-linking by crosslinker.

The GoldMag particles includes core-shell type and assembling type. The core-shell type of GoldMag particle consists of a core of magnetic material, such as Fe₃O₄, and a shell of colloidal gold coated on the surface of the core. Its particle size is about 40 nm. The assembling structure GoldMag particle is prepared by modifying magnetic core (Fe₃O₄) through silanization, and then coating colloidal gold on the surface of the core as the shell through Au—S bond. It has a particle size of about 3-5 μm.

Natural or synthetic polysaccharides have good biocompatibility without toxic effects. Moreover, they are biodegradable. These include dextran, cyclodextrin and their derivatives and so on.

The above-mentioned dextran may have a molecular weight of 10000, 20000, 30000, 40000, 50000 or 70000.

Cyclodextrin is a cyclic polysaccharide with a shape of a hollow cone with two open ends which has different diameters at each end. Cyclodextrin and its derivatives feature by particular cavities of different dimensions, and thus can form clathrates with small molecules of particular dimension and property. Cyclodextrins formed by 6, 7 or 8 glucose molecules linked by 1,4-glycosidic linkage are referred to as α-, β-or γ-cyclodextrin, respectively. Derivatives of cyclodextrin include hydroxypropyl-α-cyclodextrin, hydroxypropyl-β-cyclodextrin, hydroxypropyl-γ-cyclodextrin, methyl-β-cyclodextrin and so on.

The drug is a single agent, or a complex drug made up of two or more than two agents. It can be anti-cancer chemotherapeutic drug, protein drug, genetic drug orantibiotic drug; wherein the anti-cancer chemotherapeutic drug can be doxorubicin hydrochloride, fluorouracil, cisplatin, lobaplatin, carboplatin, methopterin and/or cytarabine; the protein drug can be a tumor-inhibiting factor; the genetic drug can be a nucleic acid vaccine; and the antibiotic drug can be aclarubicin, erythromycin or doxycycline chloridum.

The process of synthesis DPGPs is characterized:
Step 1) Preparing polysaccharide-coated GoldMag particles(PGPs)
Step 1.1) Preparing polysaccharide solution
An amount of alkaline solution with a concentration of 0.5-4 mol/L is added to polysaccharide to prepare polysaccharide solution with a concentration of 20-100 mg/ml;
Step 1.2) Synthesizing Polysaccharide-Coated GoldMag Particles
GoldMag particles and an alkaline solution with a concentration of 0.5-4 mol/L are added to the polysaccharide solution obtained from step 1.1), and the mixture reacts under stirring to obtain a suspension of polysaccharide-coated GoldMag particles; wherein the ratio of the amount of the polysaccharide used in step 1.1) to the GoldMag particles added in step 1.2) is 5-40:1;
Step 1.3) Washing
The suspension of polysaccharide-coated GoldMag particles obtained from step 1.2) is separated by magnetism and the supernatant is discarded, which process is repeated until the pH value of the solution is 7;
Step 2) Preparing DPGPs
Step 2.1) Washing
The polysaccharide-coated GoldMag particles are added to a centrifuge tube and separated by magnetism, and the supernatant is discarded;
Step 2.2) Loading Drug
A drug solution with a concentration of 0.5-1.0 mg/ml is added to the polysaccharide-coated GoldMag particles, and ultrapure water is supplemented. Then the mixture is shaked in a thermostatted shaker. After the reaction is completed, the suspension is separated by magnetism and the supernatant is discarded. The residue is freeze-dried to provide DPGPs; wherein the mass ratio of the drug to the polysaccharide-coated GoldMag particles is 1-4:20.

The mixture in the above-mentioned step 1.2) is heated to 35-45° C. before the addition of crosslinker or alkaline solution, and then the mixture is heated to 50-60° C. and the reaction time is 5-8 hours. The amount of the crosslinker or the alkaline solution added is 10%-20% in the mixture. The suspension of polysaccharide-coated GoldMag particles obtained from step 1.3) is washed by ethanol, then separated by magnetism to remove remaining organic phase. The residue is washed with ultrapure water repeatedly, until the pH value of the suspension is 7.

In the step 1.1) mentioned above, the reaction is carried out at a temperature of 20-40° C. An electric agitator can be used to accelerate the dissolution, which is operated at an appropriate speed of 300-900 revolutions per minute for 5-20 minutes. In step 1.2), the stirring is appropriately carried out at a speed of 300-900 revolutions per minute, and the reaction is preferably carried out for 4-8 hours. In step 2.1), the magnetic separation is appropriately carried out for 5-15 minutes. In step 2.2), the thermostatted shaking is preferably carried out at a temperature of 25-40° C. at a speed of 100-200 revolutions per minute for 4-20 hours, and the magnetic separation is preferably carried out for 5-15 minutes.

In the case where the drug solution in the step 2.2) mentioned above is adriamycin solution, the centrifuge tube must be wrapped with aluminium foil.

The polysaccharide mentioned above is dextran, cyclodextrin or derivatives thereof such as hydroxypropyl-β-cyclodextrin. The alkaline solution is a solution of NaOH or NH₄OH, wherein the concentration of NaOH and NH₄OH is 0.5-4 mol/L and 10-18% respectively. The crosslinker is formaldehyde, glutaraldehyde or epichlorohydrin. The drug is a single agent, or a complex drug made up of two or more than two agents. It can be a chemotherapeutic drug against cancer, a protein drug, a genetic drug or an antibiotic drug. Examples of chemotherapeutic anti-cancer drug include doxorubicin hydrochloride, fluorouracil, cisplatin, lobaplatin, carboplatin, methopterin and cytarabine. A example of the protein drug is a tumor-inhibiting factor. A example of the gene drug is a nucleic acid vaccine. Examples of the antibiotic drug include aclarubicin, erythromycin and doxycycline chloridum.

The drug and the polysaccharide-coated GoldMag particles are combined through physical adsorption without changing the structure and property of the drug. It is also possible to synthesize GoldMag particles with different particle size by adjusting conditions as desired.

### ADVANTAGES OF THE INVENTION

1. The polysaccharide-coated GoldMag particles used for drug delivery carrier have good dispersity and uniform particle size. For example, GoldMag particles coated by dextran have a uniform particle size of 220 nm and do not aggregate.

2. The polysaccharide-coated GoldMag particles used for loading drug have good biocompatibility.

3. The process of synthesizing said particles is simple and easy to operate. In addition, polysaccharide-coated GoldMag particles with different particle size can be synthesized by adjusting conditions as desired.

4. DPGPs has good effects of delaying and controlling drug release. For example, the doxorubicin loaded with dextran-coated GoldMag nanaparticles exhibit an accumulative drug release percentage in 2 hours, 24 hours and 72 hours of 18.1%, 51.4% and 77.1% in vitro, respectively.

5. The polysaccharide-coated GoldMag particles have high encapsulation rate and drug-loading rate. For example, the encapsulation rate of dextran-coated GoldMag particles is more than 93% and its drug-loading rate is more than 15.8%, both fulfilling the requirement of Chinese Pharmacopoeia.

### DESCRIPTION OF THE FIGURES

FIG. 1 The diagram of the particle size distribution of 220 nm dextran-coated GoldMag particles.

FIG. 2 The scanning electron microscope photograph of 220 nm dextran-coated GoldMag particles.

FIG. 3 The saturation magnetization value of dextran-coated GoldMag particles.

FIG. 4 Effect of adriamycin concentration on drug-loading rate (%) and encapsulation rate of dextran-coated GoldMag particles.

FIG. 5 Kinetics of doxorubicin release from adriamycin loaded dextran-coated GoldMag particles

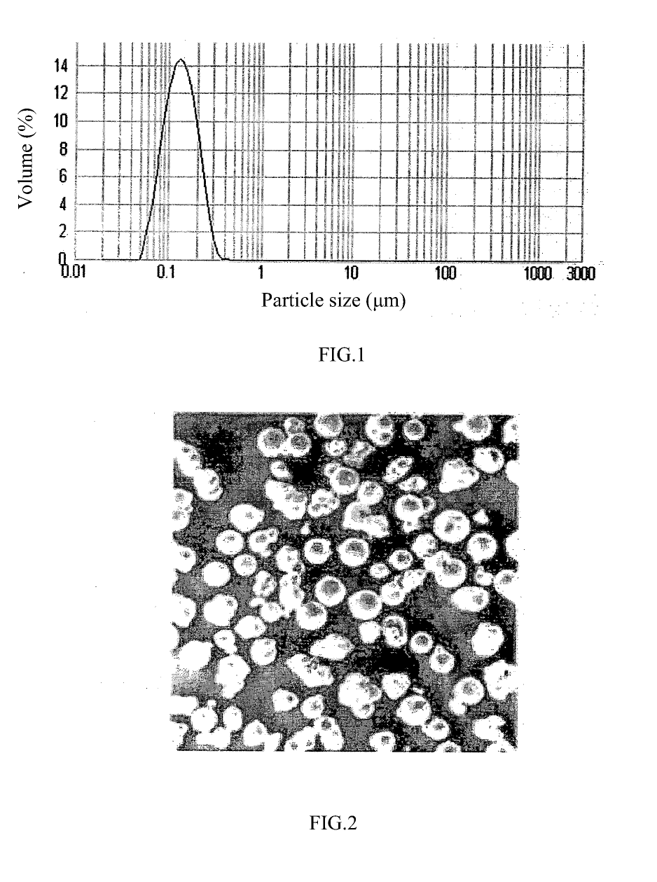

### DETAILED DESCRIPTION OF THE PREFERRED EMBODIMENT OF THE INVENTION

DPGPs is synthesized by directly mixing polysaccharide-coated GoldMag particles with drug solution to load the drug on the GoldMag particles through physical adsorption; wherein said polysaccharide-coated GoldMag particles are synthesized by mixing assembling type or core-shell type of GoldMag particles with natural or synthetic polysaccharide polymer to modify GoldMag particles with the polymer through chemical bonding or physical adsorption; or said polysaccharide-coated GoldMag particles are synthesized by using GoldMag particles as the core and make polysaccharide molecules form reticular structures through cross-linking by crosslinker.

The polysaccharide is dextran, cyclodextrin or derivatives of cyclodextrin.

The drug is a chemotherapeutic drug against cancer, a protein drug, a genetic drug or an antibiotic drug. Examples of the chemotherapeutic anti-cancer drug include doxorubicin hydrochloride, fluorouracil, cisplatin, lobaplatin, carboplatin, methopterin and cytarabine. A examples of the protein drug is a tumor-inhibiting factor. A examples of the genetic drug is a nucleic acid vaccine. Examples of the antibiotic drug include aclarubicin, erythromycin and doxycycline chloridum.

The process for preparing DPGPs includes:
Step 1) Preparing Polysaccharide-Coated GoldMag Particles
Step 1.1) Preparing a Polysaccharide Solution
An alkaline solution with a concentration of 0.5-4 mol/L is added to a polysaccharide to prepare a polysaccharide solution with a concentration of 20-100 mg/ml;
Step 1.2) Synthesizing Polysaccharide-Coated GoldMag Particles
GoldMag particles and an alkaline solution with a concentration of 0.5-4 mol/L are added to the polysaccharide solution obtained from step 1.1) to obtain a mixture. Then the obtained mixture reacts under stirring to give a suspension of polysaccharide-coated GoldMag particles; wherein the ratio of the amount of the polysaccharide used in step 1.1) to the GoldMag particles added in step 1.2) is 5-40:1;
When the polysaccharide is dextran,the mixture in the above-mentioned step 1.2) is heated to 35-45° C. before the addition of crosslinker or alkaline solution, and then the mixture is heated to 50-60° C. and the reaction time is 5-8 hours. The amount of the crosslinker or the alkaline solution added is 10%-20% in the mixture. The suspension of polysaccharide-coated GoldMag particles obtained from step 1.3) is washed by ethanol, then separated by magnetism to remove remaining organic phase, and the residue is washed with ultrapure water repeatedly, until the pH value of the suspension is 7.
Step 1.3) Washing
The suspension of polysaccharide-coated GoldMag particles obtained from step 1.2) is separated by magnetism and the supernatant is discarded, which process is repeated until the solution has a pH of 7; and
Step 2) preparing the drug-loaded body using polysaccharide-coated GoldMag particles
Step 2.1) Washing
The polysaccharide-coated GoldMag particles are placed in a centrifuge tube and separated by magnetism, and the supernatant is discarded;
Step 2.2) Loading Drug
A drug solution with a concentration of 0.5-1.0 mg/ml is added to the polysaccharide-coated GoldMag particles, and ultrapure water is supplemented. Then the mixture is shaked in a thermostatted shaker. After the reaction is completed, the suspension is separated by magnetism and the supernatant is discarded. The residue is freeze-dried to provide DPGPs; wherein the mass ratio of the drug to the polysaccharide-coated GoldMag particles is 1-4:20.

In step 1.1), the reaction is carried out at a temperature of 20-40° C. An electric agitator can be used to accelerate the dissolution, which is operated at a speed of 300-900 revolutions per minute for 5-20 minutes. In step 1.2), the stirring is carried out at a speed of 300-900 revolutions per minute, and the reaction is carried out for 4-8 hours. In step 2.1), the magnetic separation is carried out for 5-15 minutes. In step 2.2), the thermostatted shaking is carried out at a temperature of 25-40° C. at a speed of 100-200 revolutions per minute for 4-20 hours, and the magnetic separation is carried out for 5-15 minutes.

In the case where the drug solution in step 2.2) is an adriamycin solution, the centrifuge tube must be wrapped with aluminium foil.

The above-mentioned polysaccharide is dextran, cyclodextrin or derivatives of cyclodextrin. Said alkaline solution is a solution of NaOH or NH₄OH. Said crosslinker is formaldehyde, glutaraldehyde or epichlorohydrin. Said drug can be a chemotherapeutic drug against cancer, a protein drug, a genetic drug or an antibiotic drug. Examples of the chemotherapeutic drug against cancer include doxorubicin hydrochloride, fluorouracil, cisplatin, lobaplatin, carboplatin, methopterin and cytarabine. A example of the protein drug is a tumor-inhibiting factor. A example of the genetic drug is a nucleic acid vaccine. Examples of the antibiotic drug include aclarubicin, erythromycin and doxycycline chloridum.

The polysaccharide-coated GoldMag particles, as drugloaded body, exihibit high encapsulation rate and drug-loading rate. For example, superparamagnetic dextran-coated GoldMag particles have an encapsulation rate of up to more than 93% and a drug-loading rate of up to 15.9%, both fulfilling the requirement of Chinese Pharmacopoeia.

The polysaccharide-coated GoldMag particles have good effects of delaying and controlling drug release. For example, the dextran-coated GoldMag particles exhibite an accumulative drug release percentage in 2 hours, 24 hours and 72 hours of 18.1%, 51.4% and 77.1% in vitro, respectively.

The invention will be further illustrated with reference to the examples below.

### Example 1

In this example, the polysaccharide was dextran, and the drug was adriamycin.

100 mg of dextran was added into a 100 ml two-necked flask at 25° C., and 1 ml of ultrapure water and 1 ml of NaOH (1 mol/L) solution were added. An electric agitator was used to stir the mixture at a speed of 300 revolutions per minute for 10 minutes, so that dextran was thoroughly dissolved. Then, under stirring at 300 revolutions per minute, 2 ml (10 mg/ml) of core-shell type of GoldMag particles were added into the two-necked flask, followed by 2 ml (1 mol/L) of NaOH solution, and the reaction was carried out for 6 hours. After the reaction was completed, the suspension was poured into a clean beaker, and the beaker was put on a magnet (5000 gauss) to carry out magnetic separation. The supernatant was discarded. Then, another amount of ultrapure water was added and mixed homogenously. After that, magnetic separation was carried out repeatly, and the supernatant was discarded. The above operations were repeated for 3 times, until the pH value of the solution is 7. The results of observation showed that the particles had a particle size of around 2.1 μm and a saturation magnetization value of 42 emu/g.

2 mg of GoldMag particles were added To a 5 ml centrifuge tube and subjected to magnetic separation for 5 minutes. The supernatant was discarded. Then, 0.4 ml (1 mg/ml) of adriamycin solution was added, and 1.6 ml of ultrapure water was supplemented up to 2 ml. The centrifuge tube was sealed with a lid, wrapped in aluminium foil and shaked in a thermostatted shaker at 37° C. at 180 revolutions per minute. After 4 hours of reaction, the centrifuge tube was taken out and magnetic separation was carried out for 10 minutes. Then, 20 μl of the supernatant was sampled. Its absorption at 480 nm was determined by ultraviolet spectrophotometer. By calculation, it can be determined that the drug-loading rate of the dextran-coated GoldMag particles was 12.5%.

The drug-loading rate was calculated as follows:

Drug-loading rate=(total mass of adriamycin−mass of adriamycin in the supernatant)/mass of the magnetic particles×100%

### Example 2

In this example, the polysaccharide was dextran, and the drug was adriamycin.

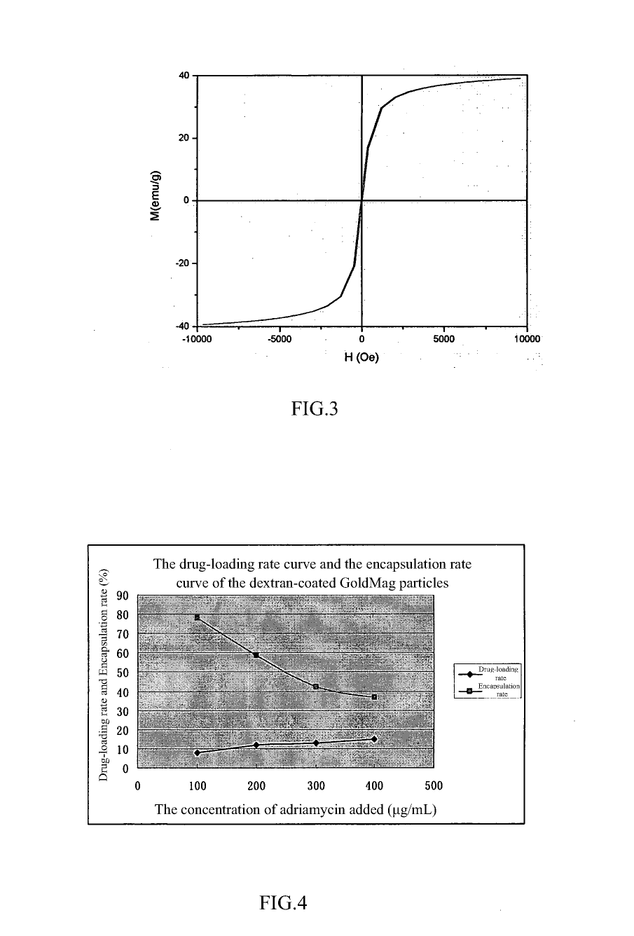

100 mg of dextran was added into a 100 ml two-necked flask at 25° C., and 2 ml (10 mg/ml) of GoldMag particles were added. An electric agitator was used to stir the mixture at a speed of 300 revolutions per minute for 10 minutes, so that dextran was thoroughly dissolved and mixed with the GoldMag particles. Then, under stirring at 300 revolutions per minute, 3 ml of NH₄OH solution having a concentration of 18% was added dropwise into the two-necked flask. The mixture was heated up to 60° C., and the reaction was carried out for 30 minutes. After the reaction was completed, the suspension was poured into a clean beaker, and the beaker was put on a magnet (5000 gauss) to carry out the magnetic separation. The supernatant was discarded. Then, an amount of ultrapure water was added and mixed homogenously. After that, magnetic separation was carried out again, and the supernatant was discarded. The above operations were repeated for 3 times, until the solution had a pH of 7. The results showed that the particles had a particle size of around 0.22 μm (cf. FIG. 1) and a saturation magnetization value of 38.8 emu/g (cf. FIG. 3).

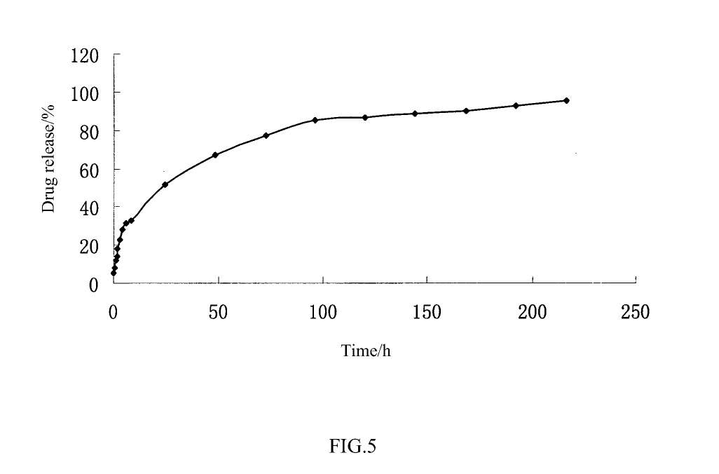

2 mg of GoldMag particles were added to a 5 ml centrifuge tube and subjected to magnetic separation for 5 minutes. The supernatant was discarded. Then, 0.4 ml (1 mg/ml) of adriamycin solution was added, and 1.6 ml of ultrapure water was supplemented up to 2 ml. The centrifuge tube was sealed with a lid, wrapped in aluminium foil and shaked in a thermostatted shaker at 37° C. at 180 revolutions per minute. After 4 hours of reaction, the centrifuge tube was taken out and magnetic separation was carried out for 10 minutes. Then, 20 μl of the supernatant was sampled. Its absorption at 480 nm was determined by ultraviolet spectrophotometer. By calculation, it can be determined that the drug-loading rate of the dextran-coated GoldMag particles was 12.05% (cf. FIG. 4).

### Example 3

In this example, the polysaccharide was dextran, the crosslinker was epichlorohydrin and the drug was adriamycin.

100 mg of dextran was added into a 100 ml two-necked flask at 25° C., and 1 ml of NaOH (1 mol/L) solution was added. An electric agitator was used to stir the mixture at a speed of 300 revolutions per minute for 10 minutes, so that dextran was thoroughly dissolved. Then, under stirring, 2 ml (10 mg/ml) of GoldMag particles were added into the two-necked flask, and the reaction was carried out for 1 hour. Next, the reaction system was heated to 40° C., and 2 ml of epichlorohydrin was added. The reaction system was further heated to 55° C. and reacted for another 6 hours. After the reaction was completed, the suspension thus obtained was poured into a clean beaker, and then the beaker was put on a magnet (5000 gauss) to carry out magnetic separation. The supernatant was discarded. Then, an amount of ethanol was added, and magnetic separation was carried out. The particles were washed for 3 times to remove remaining organic phase. Then the residue was washed with ultrapure water for 3 times, until the pH value of the solution is 7. The results showed that the particles had a particle size of around 4.2 μm and a saturation magnetization value of 48.5 emu/g.

To a 5 ml centrifuge tube, 2 mg of GoldMag particles were added and subjected to magnetic separation for 5 minutes. The supernatant was discarded. Then, 0.4 ml (1 mg/ml) of adriamycin solution was added, and 1.6 ml of ultrapure water was supplemented up to 2 ml. The centrifuge tube was sealed with a lid, wrapped in aluminium foil and shaked in a thermostatted shaker at 37° C. at 180 revolutions per minute. After 4 hours of reaction, the centrifuge tube was taken out and magnetic separation was carried out for 10 minutes. Then, 20 μl of the supernatant was sampled. Its absorption at 480 nm was determined by ultraviolet spectrophotometer. By calculation, it can be determined that the drug-loading rate of the dextran-coated GoldMag particles was 12.8%.

### Example 4

In the present example, the polysaccharide was cyclodextrin and the drug was adriamycin.

100 mg of cyclodextrin was added into a 100 ml two-necked flask at 25 ° C., and 1 ml of NaOH (1 mol/L) solution was added. An electric agitator was used to stir the mixture at a speed of 300 revolutions per minute for 10 minutes, so that cyclodextrin was thoroughly dissolved. Then, under stirring, 2 ml (10 mg/ml) of GoldMag particles were added, and the system was heated to 40° C. After that, 0.8 ml of 16.5% NH₄OH solution was added. The mixture was further heated to 50° C. and reacted for 5 hours. After the reaction was completed, the suspension thus obtained was poured into a clean beaker, and the beaker was put on a magnet (5000 gauss) to carry out magnetic separation. The supernatant was discarded. Then, an amount of ultrapure water was added and mixed homogenously. After that, magnetic separation was carried out again, and the supernatant was discarded. The above operations were repeated for 3 times, until the pH value of the solution is 7. The results showed that the particles had a particle size of around 0.32 μm (cf. FIG. 1) and a saturation magnetization value of 38.5 emu/g.

2 mg of GoldMag particles were added to a 5 ml centrifuge tube and subjected to magnetic separation for 5 minutes. The supernatant was discarded. Then, 0.4 ml (1 mg/ml) of adriamycin solution was added, and 1.6 ml of ultrapure water was supplemented up to 2 ml. The centrifuge tube was sealed with a lid, wrapped in aluminium foil and shaked in a thermostatted shaker at 37° C. at 180 revolutions per minute. After 4 hours of reaction, the centrifuge tube was taken out and magnetic separation was carried out for 10 minutes.

Then, 20 μl of the supernatant was sampled. Its absorption at 480 nm was determined by ultraviolet spectrophotometer. By calculation, it can be determined that the drug-loading rate of the cyclodextrin-coated GoldMag particles was 9.05%.

### Example 5

In this example, the polysaccharide was hydroxypropyl-β-cyclodextrin and the drug was adriamycin.

150 mg of hydroxypropyl-β-cyclodextrin was added into a 100 ml two-necked flask. An electric agitator was used to stir the mixture at a speed of 300 revolutions per minute for 10 minutes, so that hydroxypropyl-β-cyclodextrin was thoroughly dissolved. Then, under stirring, 2 ml (10 mg/ml) of GoldMag particles were added, and the system was heated to 40° C. After that, 0.8 ml of NH₄OH solution having a concentration of 16.5% was added. Under stirring, the mixture was further heated to 50° C. and reacted for 5 hours. After the reaction was completed, the dispersion of magnetic composite particles thus obtained was magnetically separated with a magnet (5000 gauss), and washed with ultrapure water repeatedly until the pH value of the supernatant is about 7. The results showed that the particles had a particle size of around 420 nm and a saturation magnetization value of 40.5 emu/g.

2 mg of GoldMag particles were added to a 5 ml centrifuge tube and subjected to magnetic separation for 5 minutes. The supernatant was discarded. Then, 0.4 ml (1 mg/ml) of adriamycin solution was added, and 1.6 ml of ultrapure water was supplemented up to 2 ml. The centrifuge tube was sealed with a lid, wrapped in aluminium foil and shaked in a thermostatted shaker at 37° C. at 180 revolutions per minute. After 4 hours of reaction, the centrifuge tube was taken out and magnetic separation was carried out for 10 minutes. Then, 20 μl of the supernatant was sampled. Its absorption at 480 nm was determined by ultraviolet spectrophotometer. By calculation, it can be determined that the drug-loading rate of the hydroxypropyl-β-cyclodextrin-coated GoldMag particles was 9.55%.

1. A drug-loaded polysaccharide-coated GoldMag particles, characterized in that it is synthesized by directly mixing polysaccharide-coated GoldMag particles with drug solution to load the drug on the GoldMag particles through physical adsorption;
wherein said polysaccharide-coated GoldMag particles are synthesized by mixing assembling or core-shell structure GoldMag particles with natural or synthetic polysaccharide polymer to coat the GoldMag particles with the polymer through chemical bonding or physical adsorption; or said polysaccharide-coated GoldMag particles are synthesized by using GoldMag particles as the core and synthesizing polysaccharide molecules reticular structure through cross-linking.

2. The drug-loaded polysaccharide-coated GoldMag particles according to claim 1, characterized in that said polysaccharide is dextran, cyclodextrin or derivatives of cyclodextrin, and said drug is a single agent or a complex drug of two or more than two agents.

3. The drug-loaded polysaccharide-coated GoldMag particles according to claim 1, characterized in that said drug is a chemotherapeutic drug against cancer, a protein drug, a genetic drug or an antibiotic drug, wherein said chemotherapeutic drug against cancer includes doxorubicin hydrochloride, fluorouracil, cisplatin, lobaplatin, carboplatin, methopterin and/or cytarabine; said protein drug includes a tumor-inhibiting factor; said genetic drug includes a nucleic acid vaccine; and said antibiotic drug can be aclarubicin, erythromycin and/or doxycycline chloridum.

4. A process for preparing the drug-loaded polysaccharide-coated GoldMag particles according to claim 1, characterized in that it includes:
Step 1) preparing polysaccharide-coated GoldMag particles
Step 1.1) preparing a polysaccharide solution
An alkaline solution with a concentration of 0.5-4 mol/L is added to polysaccharide to prepare a polysaccharide solution with a concentration of 20-100 mg/ml;
Step 1.2) synthesizing polysaccharide-coated GoldMag particles
GoldMag particles and an alkaline solution with a concentration of 0.5-4 mol/L are added to the polysaccharide solution obtained from step 1.1) to obtain a mixture, and the mixture reacts under stirring to synthesize a suspension of polysaccharide-coated GoldMag particles; wherein the ratio of the amount of the polysaccharide in step 1.1) to the GoldMag particles added in step 1.2) is 5-40:1;
Step 1.3) washing
The suspension of polysaccharide-coated GoldMag particles obtained from step 1.2) is separated by magnetism and the supernatant is discarded, which process is repeated until the solution has a pH of 7; and
Step 2) preparing drug-loaded Polysaccharide-coated GoldMag particles
Step 2.1) washing
The polysaccharide-coated GoldMag particles are placed in a centrifuge tube and separated by magnetism, and the supernatant is discarded;
Step 2.2) loading drug
To the polysaccharide-coated GoldMag particles, a drug solution with a concentration of 0.5-1.0 mg/ml is added, ultrapure water is supplemented, and the mixture is shaked in a thermostatted shaker; after the reaction is completed, magnetic separation is carried out and the supernatant is discarded; the residue is freeze-dried to provide DPGPs; wherein the ratio by mass of the drug solution added to the polysaccharide-coated GoldMag particles is 1-4:20.

5. The process for preparing the Drug-loaded Polysaccharide-coated GoldMag particles according to claim 4, characterized in that: the mixture in step 1.2) is heated to 35-45° C. before the addition of a crosslinker or alkaline solution, and then the mixture is heated to 50-60° C. and the reaction is carried out for 5-8 hours, wherein the crosslinker or alkaline solution added constitutes 10%-20% of the mixture; the suspension of polysaccharide-coated GoldMag particles obtained from step 1.3) is washed by ethanol, then separated by magnetism to remove remaining organic phase, and the residue is washed with ultrapure water repeatedly, until the pH value of the solution is 7.

6. The process for preparing drug-loaded Polysaccharide-coated GoldMag particles according to claim 4 or 5, characterized in that: in step 1.1), the reaction is carried out at a temperature of 20-40° C., and an electric agitator can be used to accelerate the dissolution, which is operated at a speed of 300-900 revolutions per minute for 5-20 minutes; in step 1.2), the stirring is carried out at a speed of 300-900 revolutions per minute, and the reaction is carried out for 4-8 hours; in step 2.1), the magnetic separation is carried out for 5-15 minutes; and in step 2.2), the thermostatted shaking is carried out at a temperature of 25-40° C. at a speed of 100-200 revolutions per minute for 4-20 hours, and the magnetic separation is carried out for 5-15 minutes.

7. The process for preparing drug-loaded Polysaccharide-coated GoldMag particles according to claim 6, characterized in that: when the drug solution in step 2.2) is adriamycin solution, the centrifuge tube is wrapped with aluminum foil.

8. The process for preparing Drug-loaded Polysaccharide-coated GoldMag particles according to claim 7, characterized in that: said polysaccharide is dextran, cyclodextrin or hydroxypropyl-β-cyclodextrin; said alkaline solution is a solution of NaOH or NH₄OH; said crosslinker is formaldehyde, glutaraldehyde or epichlorohydrin; and said drug is a single agent or a complex drug of two or more than two agents.

9. The process for preparing Drug-loaded Polysaccharide-coated GoldMag particles according to claim 8, characterized in that said drug is a chemotherapeutic drug against cancer, a protein drug, a genetic drug or an antibiotic drug, wherein said anti-cancer chemotherapeutic drug include doxorubicin hydrochloride, fluorouracil, cisplatin, lobaplatin, carboplatin, methopterin and/or cytarabine; said protein drug includes a tumor-inhibiting factor; said genetic drug includes a nucleic acid vaccine; and said antibiotic drug includes aclarubicin, erythromycin and/or doxycycline chloridum.

10. The process for preparing drug-loaded Polysaccharide-coated GoldMag particles according to claim 5, characterized in that: in step 1.1), the reaction is carried out at a temperature of 20-40° C., and an electric agitator can be used to accelerate the dissolution, which is operated at a speed of 300-900 revolutions per minute for 5-20 minutes; in step 1.2), the stirring is carried out at a speed of 300-900 revolutions per minute, and the reaction is carried out for 4-8 hours; in step 2.1), the magnetic separation is carried out for 5-15 minutes; and in step 2.2), the thermostatted shaking is carried out at a temperature of 25-40° C. at a speed of 100-200 revolutions per minute for 4-20 hours, and the magnetic separation is carried out for 5-15 minutes.

11. The process for preparing drug-loaded Polysaccharide-coated GoldMag particles according to claim 10, characterized in that: when the drug solution in step 2.2) is adriamycin solution, the centrifuge tube is wrapped with aluminum foil.

12. The process for preparing Drug-loaded Polysaccharide-coated GoldMag particles according to claim 11, characterized in that: said polysaccharide is dextran, cyclodextrin or hydroxypropyl-β-cyclodextrin; said alkaline solution is a solution of NaOH or NH₄OH; said crosslinker is formaldehyde, glutaraldehyde or epichlorohydrin; and said drug is a single agent or a complex drug of two or more than two agents.

13. The process for preparing Drug-loaded Polysaccharide-coated GoldMag particles according to claim 12, characterized in that said drug is a chemotherapeutic drug against cancer, a protein drug, a genetic drug or an antibiotic drug, wherein said anti-cancer chemotherapeutic drug include doxorubicin hydrochloride, fluorouracil, cisplatin, lobaplatin, carboplatin, methopterin and/or cytarabine; said protein drug includes a tumor-inhibiting factor; said genetic drug includes a nucleic acid vaccine; and said antibiotic drug includes aclarubicin, erythromycin and/or doxycycline chloridum.
